(19) United States
(12) Patent Application Publication
(10) Pub. No.: US 2023/0045494 A1
(43) Pub. Date: Feb. 9, 2023

(54) COMBINATION THERAPIES BASED ON CTLA4 AND IL-17B INHIBITORS

(71) Applicant: OREGA BIOTECH, ECULLY (FR)

(72) Inventors: Nathalie BONNEFOY, SAINT CLEMENT DE RIVIERE (FR); Jérémy BASTID, TASSIN LA DEMI LUNE (FR); Armand BENSUSSAN, PARIS (FR)

(21) Appl. No.: 17/792,631

(22) PCT Filed: Feb. 25, 2021
(86) PCT No.: PCT/EP2021/054744

§ 371 Date: Jul. 13, 2022

(30) Foreign Application Priority Data
Feb. 28, 2020 (EP) 20305206.3

(51) Int. Cl. C07K 16/28; C07K 16/24; A61P 35/00

(52) U.S. Cl. CPC C07K 16/2818; C07K 16/244; A61P 35/00; C07K 2317/24; C07K 2317/76

(57) ABSTRACT

The present invention concerns the combination of CTLA4 and IL-17B inhibitors, especially for the treatment of patients and diseases resistant to anti-CTLA4 therapies.

### FIELD OF THE INVENTION

The present invention relates to combination therapies based on CTLA4 and IL-17B inhibitors.

### BACKGROUND OF THE INVENTION

Immune checkpoints are regulators of the immune system. These pathways are crucial for self-tolerance, which prevents the immune system from attacking cells indiscriminately.

The chronicity of cancer and persistent infections provides constant antigen exposure to antigen-reactive T-cells, leading to cellular exhaustion and abrogation of effector functions. The expression of cell surface-bound molecules such as programmed cell death protein 1 (PD1/PD-1/CD279) and cytotoxic T lymphocyte-associated protein 4 (cytotoxic T lymphocyte-associated antigen 4/CTLA4/CTLA-4/CD152) on antigen-specific T-cells are markers of exposure to immunogenic stimuli. PD1 and CTLA4 identify as immune checkpoints due to their crucial role in down-regulating the magnitude of T-cell responses. Other immune checkpoint molecules of clinical relevance are the T-cell immunoglobulin and mucin-domain containing 3 (TIM3/TIM-3), with its nominal ligand galectin 9, and to a lesser extent the lymphocyte-activation gene 3 (LAG3/LAG-3/CD223), which binds to major histocompatibility complex (MHC) class II molecules with higher affinity than the CD4 receptor.

CTLA-4 expressed on antigen-specific T-cells is an essential modulator of T cell activation. T cell receptor (TCR) binding to the major histocompatibility complex (MHC) on the surface of an antigen-presenting cell (APC) provides specificity to T-cell activation, but further costimulatory signals are required. Binding of B7-1 (CD80) or B7-2 (CD86) molecules on APC with CD28 molecules on the T cell leads to signaling within the T cell associated with proliferation of T cells, increased T-cell survival, and differentiation through the production of growth cytokines such as interleukin-2 (IL-2). CTLA-4 is a CD28 homolog with much higher binding affinity for B7 molecules. Unlike CD28, binding of CTLA-4 to B7 does not produce a stimulatory signal but produces inhibitory signals that counteract the stimulatory signals from CD28:B7 and TCR:MHC binding. Proposed mechanisms for such inhibitory signals include direct inhibition at the TCR immune synapse, inhibition of CD28 or its signaling pathway, or increased mobility of T cells leading to decreased ability to interact with APCs (for review see Collins et al., Immunity (2002), 17(2):201-10; Egen et al., Nat Immunol. (2002), 3(7):611-8). Fatal autoimmunity in CTLA-4 knockout mice resulting from the release of self-reactive T cells illustrates that CTLA-4 is a critical negative regulator of T cell responses (Tivol et al., Immunity (1995), 3(5):541-7; Waterhouse et al., Science (1995), 270(5238):985-8; Ise et al., Nat Immunol. (2010), 11 (2): 129-35).

CTLA-4 itself is subject to regulation, particularly by localization within the cell. In resting naïve T cells CTLA-4 is located primarily in the intracellular compartment. Stimulatory signals resulting from both TCR and CD28:B7 binding induce upregulation of CTLA-4 on the cell surface by exocytosis of CTLA-4-containing vesicles.

Unlike effector T cells, regulatory T cells (Tregs) constitutively express CTLA-4, and this is thought to be important for their suppressive functions. In animal models, genetic CTLA-4 deficiency in Tregs impaired their suppressive functions (Takahashi T et al., J Exp Med (2000), 192(2):303-10).

By targeting factors that foster the development and maintenance of an immunosuppressive microenvironment, e.g. within tumors, immunotherapies can release the brakes on the host's own immune system and possibly cure of disease.

Initially, murine models demonstrated that anti-CTLA-4 antibody treatments resulted in tumor regression (Leach et al., Science (1996), 271(5256):1734-6). Based upon the efficacy of CTLA-4 blockade in animal models, anti-CTLA-4 antibodies were developed for clinical use.

In practice, a first checkpoint inhibitor (CPi) targeting CTLA4 has been developed: Ipilimumab (YERVOY®), a fully humanized monoclonal antibody anti-CTLA4. Anti-CTLA-4 blockade with ipilimumab was the first treatment to prolong overall survival in patients with advanced melanoma in a randomized setting (Hodi et al., N Engl J Med (2010), 363(8):711-23; Robert et al., N Engl J Med (2011), 364(26):2517-26). Analysis of long-term survival data pooled across several phase II and phase III trials showed that the survival curve begins to plateau at about 3 years, with 3-year survival rates of 22%, 26%, and 20% in all patients with sufficient follow-up, in treatment-naïve patients, and in previously treated patients, respectively (Schadendorf et al., J clin Oncol (2015), 33(17):1889-94). Consistent with its survival benefit, CTLA-4 blockade is associated with durable responses in a proportion of patients treated, with some responses reported to last >3 years (Hodi et al., N Engl J Med (2010), 363(8):711-23; Farolfi et al., Melanoma Res (2012), 22(3):263-70). Alongside the benefits in tumour control, these trials nonetheless demonstrate a broad range of immune related adverse events (irAEs) occurring in 60-65% of patients. irEAs most commonly effect the skin, gastrointestinal (GI) tract, liver and endocrine organs (for review see Boutros et al., Nat Rev Clin Oncol. (2016), 13(8):473-86).

Ipilimumab (YERVOY®) has been authorized in Europe and in the United States for treating melanoma, especially unresectable or metastatic melanoma. Moreover and for a better efficiency, it can be used in combination with Nivolumab (Opdivo®) for treating melanoma, renal cell carcinoma (RCC) and colorectal cancer (CRC).

Tremelimumab (formerly ticilimumab, CP-675,206) is a fully human IgG2 monoclonal antibody against CTLA-4, i.e. an immune checkpoint blocker. Previously in development by Pfizer, it is now in investigation by MedImmune. It has been undergoing human trials for the treatment of various cancers but has not attained approval yet.

Other anti-CTLA4 antibodies in development include for example the Fc-engineered recombinant human IgG1 anti-CTLA-4 monoclonal antibody AGEN1181, the fully human IgG1 anti-CTLA-4 monoclonal antibody zalifrelimab (AGEN1884), the humanized IgG1 anti-CTLA-4 monoclonal antibody ONC-392, the anti-CTLA-4 monoclonal antibody CS1002, the fully human anti-CTLA-4 monoclonal antibody IBI-310, the fully human IgG1 anti-CTLA-4 monoclonal antibody REGN4659. Other antibodies of interest may include AK104, a PD-1 and CTLA-4 bispecific antibody or BCD-217, a combination of anti-CTLA-4 and anti-PD-1 monoclonal antibodies.

Besides, as the phenomenon of T-cell exhaustion in chronic infections is similar to what observed in cancer, the same strategy has been applied to chronic infectious diseases such as human immunodeficiency virus (HIV), simian immunodeficiency virus (SIV), hepatitis (HBV) and malaria (for a review, see Wykes et al., Nat Rev Immunol. (2018), 18(2):91-104).

However, in view of the limited efficacy of the anti-CTLA4 based therapy and known toxicity of the checkpoint inhibitors, there is still a need to develop more efficient strategies for treating said kind of diseases.

### SUMMARY OF THE INVENTION

The present invention relates to combination therapies for the treatment of e.g. cancer or infectious diseases. In particular, the present invention is defined by the claims.

### DETAILED DESCRIPTION OF THE INVENTION

Cytotoxic T lymphocyte-associated protein 4 (cytotoxic T lymphocyte-associated antigen 4/CTLA4/CTLA-4/CD152) is a possible therapeutic target for various types of cancer. However, some tumors are refractory to anti-CTLA4. In the present application, the inventors reveal the link between the CTLA4 immune checkpoint and IL-17B. They found that the simultaneous inhibition of CTLA4 and IL-17B is efficient versus different types of cancer. Collectively, the data reveal the first evidence indicating the importance of IL-17B for resistance against anti-CTLA4 therapeutics and suggest blocking IL-17B in combination with anti-CTLA4 as a novel therapeutic strategy to combat diseases involving the CTLA4 immune checkpoint.

Accordingly, the first object of the present invention relates to a composition comprising a CTLA4 inhibitor and an IL-17B inhibitor.

According to a specific embodiment, a composition according to the invention herein has an amount of the CTLA4 inhibitor inferior to its amount in a composition not comprising an IL-17B inhibitor.

According to another aspect, the invention relates to the use of such a composition in therapy, advantageously for the treatment of a cancer or an infectious disease as detailed below.

According to a second aspect and advantageously in relation to the treatment of a cancer or an infectious disease, the invention concerns a composition comprising an IL-17B inhibitor for use in administering a subject treated with a CTLA4 inhibitor. According to a specific embodiment, the subject is resistant to the treatment with the CTLA4 inhibitor. In other words, the invention also relates to a composition comprising an IL-17B inhibitor for use in increasing the sensitivity of a subject to a CTLA4 inhibitor.

As used therein, the expression “resistant to CTLA4 inhibitors” can refer to the fact that:
The majority of the patients having a given disease do not respond to these treatments (CTLA4 inhibitors) and/or have a poor prognostic. In that case, the disease is considered to be globally resistant to anti-CTLA4 treatments, whereas other diseases are sensitive to said treatments. As an example, certain types of cancer are known to be more resistant than others to CTLA4 inhibitors; and/or
Even if a disease is globally known to be sensitive to anti-CTLA4 treatments, a given patient having said disease can be resistant to anti-CTLA4 therapies. This wording covers primary resistance to said therapies as well as acquired resistance to said therapies as defined above. According to another embodiment, a patient being resistant can be a patient with hyperprogressive disease (HPD) following anti-CTLA4 treatment, i.e. with accelerated disease upon treatment with CTLA4 inhibitors.

A further aspect of the invention concerns a method for enhancing the potency of a CTLA4 inhibitor administered to a patient as part of a treatment regimen, the method comprising administering to the subject a pharmaceutically effective amount of an IL-17B inhibitor in combination with the CTLA4 inhibitor. In other words, the invention concerns a composition comprising an IL-17B inhibitor for use in increasing the efficacy of a treatment with a CTLA4 inhibitor in a subject. It further concerns a method of treating a patient in need thereof comprising administering to the patient a therapeutically effective combination of a CTLA4 inhibitor with an IL-17B inhibitor, wherein administration of the combination results in enhanced therapeutic efficacy relative to the administration of the CTLA4 inhibitor alone.

As used herein, the expression “enhancing the potency or efficacy of a CTLA4 inhibitor” refers to the ability of the IL-17B inhibitor to increase the ability of the CTLA4 inhibitor to inhibit the progression of the disease and then to improve the therapeutic outcome.

In the frame of the invention, the treatment is advantageously dedicated to a disease involving the CTLA4 immune checkpoint. As previously reported in the literature, said disease is advantageously selected in the group consisting of: cancer and infectious diseases, especially chronic infections. An infectious disease is advantageously selected in the following group: severe sepsis, septic shock, viral infections especially infections by human immunodeficiency virus (HIV), simian immunodeficiency virus (SIV), hepatitis virus, particularly hepatitis B virus (HBV) and hepatitis C virus (HCV), cytomegalovirus or Epstein-Barr virus, fungal infections such as mucormycosis, mosquito-borne infectious diseases such as malaria, and bacterial infections such as tuberculosis (TB).

As used herein and as illustrated in relation to cancer, the expression “enhanced therapeutic efficacy” refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, a delayed tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition comprising the CTLA4 inhibitor with the IL-17B inhibitor to the activity and/or efficacy of the CTLA4 inhibitor alone, refers to a comparison using amounts known to be comparable according to one of skill in the art.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, oesophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; undifferentiated carcinoma; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods and compositions of the present invention are particularly suitable for the treatment of the following cancers: melanoma, especially unresectable or metastatic melanoma; Renal Cell Carcinoma (RCC); ColoRectal Cancer (CRC), especially colon cancer. Further cancers to be treated in the frame of the invention include: Small Cell Lung Cancer (SLC), Non-Small Cell Lung Cancer (NSCLC), esophageal cancer, breast cancer, Hepatocellular Carcinoma (HCC), thyroid cancer, pancreatic cancer, ovarian cancer, Myelodysplastic Syndrome, Acute Myeloid Leukemia, Diffuse Large B-Cell Lymphoma, glioblastoma, sarcoma, Soft tissue Sarcoma, nasopharyngeal carcinoma, mesothelioma, head and neck cancer, prostate cancer, gastrointestinal cancer.

In some embodiments, methods and compositions of the present invention are particularly suitable for the treatment of diseases, especially cancers, resistant to CTLA4 inhibitors. As used herein, the term “resistant” refers to the repeated outbreak of the disease, or a progression of the disease independently of whether the disease was cured before said outbreak or progression.

“Antineoplastic resistance” is the drug resistance of neoplastic (cancerous) cells, or the ability of cancer cells to survive and grow despite anti-cancer therapies. There are two general causes of antineoplastic therapy failure: Inherent properties, such as genetic characteristics, giving cancer cells their resistance, which is rooted in the concept of cancer cell heterogeneity and acquired resistance after drug exposure. Cancer cells can become resistant to drugs by various mechanisms, including: altered membrane transport, enhanced DNA repair, apoptotic pathway defects, alteration of target molecules, protein and pathway mechanisms, such as enzymatic deactivation. Since cancer is a genetic disease, two genomic events underlie these mechanisms of acquired drug resistance: Genome alterations (e.g. gene amplification and deletion) and epigenetic modifications (Housman et al., Cancer, 2014, 6, 1769-1792).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the “CTLA4” has its general meaning in the art and refers to as a cytotoxic T lymphocyte-associated protein 4. As used herein, the terms “cytotoxic T lymphocyte-associated protein 4”, “cytotoxic T lymphocyte-associated antigen 4”, “CTLA4”, “CTLA-4”, and “CD152” are used interchangeably. By “CTLA4 ligand” is meant a polypeptide which binds to and/or activates CTLA4, especially B7-1 (also named CD80) or B7-2 (also named CD86).

As used herein the term “CTLA4 inhibitor” refers to an agent which interferes with CTLA4 activation or function. Examples of CTLA4 inhibitors include CTLA4 antibodies (e.g. anti-CTLA4, anti-B7-1 or anti-B7-2 antibodies); CTLA4-Ig, CD80-Ig, CD86-Ig; organic molecule antagonists; and/or agents that bind to, or interfere with function of CTLA4. Typically, the CTLA4 inhibitor is an antibody or small organic molecule which binds to the CTLA4 receptor or to its ligand B7-1 or B7-2.

In some embodiments, the CTLA4 inhibitor is a small organic molecule. As used herein, the term “small organic molecule” refers to a molecule of size comparable to those organic molecules generally used in pharmaceuticals. The term excludes biological macromolecules (e.g.; proteins, nucleic acids, etc.); preferred small organic molecules range in size up to 2000 Da, and most preferably up to about 1000 Da.

Patent publications related to CTLA4 antibodies include: WO00/37504; WO2006/029219; WO2016/130898; WO2018/156250.

Examples of CTLA4 antibodies include Ipilimumab (YERVOY®) already used in therapy, especially in cancer therapy, and tremelimumab (formerly ticilimumab, CP-675,206), as well as antibody AGEN1181, zalifrelimab (AGEN1884), ONC-392, CS1002, IBI-310 and REGN4659. Other antibodies of interest may include AK104, a PD-1 and CTLA-4 bispecific antibody or BCD-217, or a combination of anti-CTLA-4 and anti-PD-1 monoclonal antibodies. According to a specific embodiment, CTLA4 antibodies are used in combination with PD1 antibodies. According to a preferred embodiment Ipilimumab (YERVOY®) is used in combination with Nivolumab (Opdivo®).

The interleukin 17 (IL-17) family comprises 6 interleukins (IL-17A, IL-17B, IL-17C, IL-17D, IL-17E and IL-17F) and their receptors (IL-17RA, IL-17RB, IL-17RC, IL-17RD and IL-17RE) (Gaffen, S. L. (2009) Nature reviews. Immunology 9(8): 556-567). IL-17B binds the dimeric IL-17RB receptor and IL-17E binds a complex of IL-17RA and IL-17RB.

As used herein the term “IL-17B” has its general meaning in the art and a polypeptide having a sequence according to GenBank Acc. No. NP 001304916.1 or NP 055258.1, the product of the human IL-17B gene, and include all of the variants, isoforms or species homologs of IL-17B. As used herein, the term “IL-17B signaling” as used herein means the processes initiated by IL-17B or a second IL-17B receptor ligand interacting with the IL-17RB receptor on the cell surface, resulting in measurable changes in cell function. Typically, IL-17B signaling can be assessed by functional assays measuring for example effect of IL-17B receptor ligand on cell proliferation or differentiation, or using reporter genes and reporter gene constructs.

As used herein, the term “IL17RB” (IL-17RB, CRL4, EVI27, IL17RH1, or MGC5245) as used herein means “interleukin 17 receptor B”, a polypeptide having an amino acid sequence according to GenBank Acc. No. NP061195, the product of the human IL17RB receptor gene, and include all of the variants, isoforms and species homologs of IL17RB.

Accordingly, as used herein the terms “IL-17B inhibitors” refers to any compound that is able to inhibit the IL-17B signaling. The IL-17B inhibitor to be used in the methods and compositions described herein is a molecule that blocks, suppresses, or reduces (including significantly) the biological activity of the IL-17B cytokine, including downstream pathways mediated by IL-17B signaling. Thus the term “IL-17B inhibitor” implies no specific mechanism of biological action whatsoever, and is deemed to expressly include and encompass all possible pharmacological, physiological, and biochemical interactions with IL-17B whether direct or indirect.

In some embodiments, the IL-17B inhibitor is selected from the group consisting of antibodies directed against IL-17B and antibodies directed against a receptor of IL-17B (e.g., an antibody specifically binds IL-17RB or the dimeric complex formed thereby). According to a specific embodiment, the IL-17RB inhibitor is an IL-17E inhibitor, e.g. an antibody that specifically binds IL-17E.

Patent publications related to IL17B/IL17RB antibodies include: WO2010/116123, WO2011/044563, WO2016/004045, US2009/0291097.

IL17B/IL17RB antibodies are commercially available, e.g. the mouse IL-17B antibody (AF1709; R&D Systems) or the human IL-17RB antibody (MAB1207; R&D Systems).

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In some embodiments, the antibody does not comprise an Fc portion that induces antibody dependent cellular cytotoxicity (ADCC). The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551.

According to a further embodiment, the inhibitory activity against CTLA4 and IL-17B is harboured by a single molecule, i.e. a bispecific inhibitor. According to a specific embodiment, such an inhibitor is a bispecific antibody which comprises a first antigen-binding domain that binds to CTLA4 and a second antigen-binding domain that binds to IL-17B or IL-17RB or IL-17E.

In some embodiments, the CTLA4 or IL-17B inhibitor is an inhibitor of CTLA4, B7-1 or B7-2, IL-17B or IL-17RB expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of CTLA4 or IL-17B mRNA by binding thereto and thus preventing protein translation or increasing mRNA degradation, thus decreasing the level of CTLA4 or IL-17B, and thus activity, in a cell. For example, antisense oligonucleotides of at least about 15 bases and complementary to unique regions of the mRNA transcript sequence encoding CTLA4 or IL-17B can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. CTLA4 or IL-17B gene expression can be reduced by contacting a patient or cell with a small double stranded RNA (dsRNA), or a vector or construct causing the production of a small double stranded RNA, such that CTLA4 or IL-17B gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing CTLA4 or IL-17B. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

As used herein the term “co-administering” as used herein means a process whereby the combination of the IL-17B inhibitor and the CTLA4 inhibitor is administered to the same patient. The IL-17B inhibitor and the CTLA4 inhibitor may be administered simultaneously, at essentially the same time, or sequentially. The IL-17B inhibitor and the CTLA4 inhibitor need not be administered by means of the same vehicle. The IL-17B inhibitor and the CTLA4 inhibitor may be administered one or more times and the number of administrations of each component of the combination may be the same or different. In addition, the IL-17B inhibitor and the CTLA4 inhibitor need not to be administered at the same site.

As used herein, the term “therapeutically effective combination” as used herein refers to an amount or dose of an IL-17B inhibitor together with the amount or dose of the CTLA4 inhibitor that is sufficient to treat the disease, especially cancer. The amount of the IL-17B inhibitor in a given therapeutically effective combination may be different for different individuals and different tumor types, and will be dependent upon the one or more additional agents or treatments included in the combination. The “therapeutically effective amount” is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the IL-17B inhibitor and the CTLA4 inhibitor are administered to the patient in the form of a pharmaceutical composition. Typically, the IL-17B inhibitor and the CTLA4 inhibitor may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The IL-17B inhibitor and the CTLA4 inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the patient being treated. The person responsible for administration will, in any event, determine the appropriate dose for the individual patient.

According to other embodiments, the administration of the CTLA4 and IL-17B inhibitors is combined with other treatments dedicated to the same disease. In case the further treatment also corresponds to the administration of a given molecule, said molecule can be present in the same composition as the one containing the CTLA4 inhibitor and/or the IL-17B inhibitor, or can be administered separately.

In relation to cancer, the other treatment can be e.g.:
local surgery;
surgery;
radiation or radiotherapy;
chemotherapy;
immunotherapy;
targeted therapy, e.g. using BRAF/MEK inhibitors;
hormone therapy;
stem cell transplant;
precision medicine;
antitumor antibodies;
gene therapy;
vaccine;
cell therapy;
CAR (chimeric antigen receptor)-T cell therapy;
TCR (T cell receptor) therapy;
induction therapy;
consolidation therapy
maintenance therapy;
differentiating agents;
angiogenesis inhibitors.

Further immunotherapy encompasses other checkpoint inhibitors (CPi) e.g. targeting PD1, LAG3, TIM3 and/or TIGIT.

Patent publications related to PD1/PDL1 antibodies include: WO2018/204303, CN108640992, WO2018/036472, CN107384933, WO2015/035606, CN107043425, CN106939050, CN106749663.

A non-exhaustive list of PD1/PDL1 antibodies comprises: Pembrolizumab (Keytruda®), Nivolumab (Opdivo®), BMS-936559 (MDX 1105), Cemiplimab (REGN2810), Cemiplimab-rwlc (LIBTAYO®), Avélumab (MSB0010718C or Bavencio), Durvalumab (MEDI4736 or INFIMZI®), Atezolizumab (MPDL3280A or Tecentriq®), Spartalizumab (PDR 001), as well as their combination.

Other immune-oncology (TO) agents include those targeting OX40, GITR, ICOS, VISTA, CD39, CD40, CD47, CD70 (e.g. anti mAbs ARGX-110 and MDX-1203), CD73 or CD137.

Vaccines are also further possible treatments, especially vaccines having PRR (Pattern Recognition Receptors such as Toll-Like Receptors or TLR) agonist properties, e.g. vaccines based on attenuated rotavirus, reovirus or on Newcastle Disease Virus (NDV).

Chemotherapeutic agents to be used with the combination according to the invention include vinca alkaloids, epipodophyllotoxins, anthracycline antibiotics, actinomycin D, plicamycin, puromycin, gramicidin D, paclitaxel (Taxol™, Bristol Myers Squibb), colchicine, cytochalasin B, emetine, maytansine, and amsacrine (or “mAMSA”). The vinca alkaloid class is described in GOODMAN AND GILMAN'S THE PHARMACOLOGICAL BASIS OF THERAPEUTICS (7th ed.), (1985), pp. 1277-1280. Exemplary of vinca alkaloids are vincristine, vinblastine, and vindesine. The epipodophyllotoxin class is described, for example, in GOODMAN AND GILMAN'S THE PHARMACOLOGICAL BASIS OF THERAPEUTICS (7th ed.), (1985), pp. 1280-1281. Exemplary of epipodophyllotoxins are etoposide, etoposide orthoquinone, and teniposide. The anthracycline antibiotic class is described in GOODMAN AND GILMAN'S THE PHARMACOLOGICAL BASIS OF THERAPEUTICS (7th ed.), (1985), pp. 1283-1285. Exemplary of anthracycline antibiotics are daunorubicin, doxorubicin, mitoxantraone, and bisanthrene. Actinomycin D, also called Dactinomycin, is described, for example, in GOODMAN AND GILMAN'S THE PHARMACOLOGICAL BASIS OF THERAPEUTICS (7th ed.), (1985), pp. 1281-1283. Plicamycin, also called mithramycin, is described in Goodman and Gilman's The Pharmacological Basis of Therapeutics (7th ed), (1985), pp. 1287-1288. Additional chemotherapeutic agents include cisplatin (Platinol™, Bristol Myers Squibb), carboplatin (Paraplatin™, Bristol Myers Squibb), mitomycin (Mutamycin™, Bristol Myers Squibb), altretamine (Hexalen™, U.S. Bioscience, Inc.), cyclophosphamide (Cytoxan™, Bristol Myers Squibb), lomustine (CCNU) (CeeNU™ Bristol Myers Squibb), carmustine (BCNU) (BiCNU™, Bristol Myers Squibb).

Exemplary chemotherapeutic agents also include aclacinomycin A, aclarubicin, acronine, acronycine, adriamycin, aldesleukin (interleukin-2), altretamine (hexamiethylmelamine), aminoglutethimide, aminoglutethimide (cytadren), aminoimidazole carboxamide, amsacrine (m-AMSA; amsidine), anastrazole (arimidex), ancitabine, anthracyline, anthramycin, asparaginase (elspar), azacitdine, azacitidine (ladakamycin), azaguanine, azaserine, azauridine, 1,1′,1″-phosphinothioylidynetris aziridine, azirino(2′, 3′:3,4)pyrrolo(1,2-a)indole-4,7-dione, BCG (theracys), BCNU, BCNU chloroethyl nitrosoureas, benzamide, 4-(bis(2-chloroethyl)amino)benzenebutanoic acid, bicalutamide, bischloroethyl nitrosourea, bleomycin (blenozane), bromodeoxyuridine, broxuridine, busulfan (myleran), carbamic acid ethyl ester, chlorambucil (leukeran), chloroethyl nitrosoureas, chorozotocin (DCNU), chromomycin A3, cis-retinoic acid, cladribine (2-chlorodeoxyadenosine; 2cda; leustatin), coformycin, cycloleucine, cyclophosphamide anhydrous, chlorambucil, cytarabine, cytarabine, cytarabine HCl (cytosar-u), 2-deoxy-2-(((methylnitrosoamino)carbonyl)amino)-D-glucose, dacarbazine, decarbazine, decarbazine (DTIC-dome), demecolcine, dexamethasone, dianhydrogalactitol, diazooxonorleucine, diethylstilbestrol, docetaxel (taxotere), eflomithine, estramustine, estramustine phosphate sodium (emcyt), ethiodized oil, ethoglucid, ethyl carbamate, ethyl methanesulfonate, fenretinide, floxuridine, floxuridine (fudr), fludarabine (fludara), fluorouracil (5-FU), fluoxymesterone (halotestin), flutamide, flutamide (eulexin), fluxuridine, gallium nitrate (granite), gemcitabine (gemzar), geni stein, 2-deoxy-2-(3-methyl-3-nitrosoureido)-D-glucopyranose, goserelin (zoladex), hexestrol, hydroxyurea (hydra), idarubicin (idamycin), ifosfagemcitabine, ifosfamide (iflex), ifosfamide with mesna (MAID), interferon, interferon alfa, interferon alfa-2a, alfa-2b, alfa-n3, interleukin-2, iobenguane, iobenguane iobenguane, irinotecan (camptosar), isotretinoin (accutane), ketoconazole, 4-(bis(2-chloroethyl)amino)-L-phenylalanine, L-serine diazoacetate, lentinan, leucovorin, leuprolide acetate (LHRH-analog), levamisole (ergamisol), mannomustine, maytansine, mechlorethamine, mechlorethamine HCl (nitrogen mustard), medroxyprogesterone acetate (provera, depo provera), megestrol acetate (menace), melengestrol acetate, melphalan (alkeran), menogaril, mercaptopurin, mercaptopurine (purinethol), mercaptopurine anhydrous, MESNA, mesna (mesne), methanesulfonic acid, ethyl ester, methotrexate (mtx; methotrexate), methyl-ccnu, mimosine, misonidazole, mithramycin, mitoantrone, mitobronitol, mitoguazone, mitolactol, mitomycin (mutamycin), mitomycin C, mitotane (o,p′-DDD; lysodren), mitoxantrone HCl (novantrone), mopidamol, N,N-bis(2-chloroethyl)tetrahydro-2H-1,3,2-oxazaphosphorin-2-amine-2-oxide, N-(1-methylethyl)-4-((2-methylhydrazino)methyl)benzamide, N-methyl-bis(2-chloroethyl) amine, nicardipine, nilutamide (nilandron), nimustine, nitracrine, nitrogen mustard, nocodazole, nogalamycin, octreotide (sandostatin), pactamycin, pegaspargase (PEGx-1), pentostatin (2′-deoxycoformycin), peplomycin, peptichemio, photophoresis, picibanil, pipobroman, podofilox, podophyllotoxin, porfiromycin, prednisone, procarbazine, procarbazine HCl (matulane), prospidium, puromycin aminonucleoside, PUVA (psoralen+ultraviolet a), pyran copolymer, rapamycin, s-azacytidine, 2,4,6-tris(1-aziridinyl)-s-triazine, semustine, showdomycin, sirolimus, streptozocin (zanosar), suramin, tamoxifen citrate (nolvadex), taxon, tegafur, tenuazonic acid, TEPA, testolactone, thio-tepa, thioguanine, thiotepa (thioplex), tilorone, topotecan, tretinoin (vesanoid), triaziquone, trichodermin, triethylene glycol diglycidyl ether, triethylenemelamine, triethylenephosphoramide, triethylenethiophosphoramide, trimetrexate (neutrexin), tris(1-aziridinyl)phosphine oxide, tris(1-aziridinyl)phosphine sulfide, tris(aziridinyl)-p-benzoquinone, tris(aziridinyl)phosphine sulfide, uracil mustard, vidarabine, vidarabine phosphate, vinorelbine, vinorelbine tartrate (navelbine), (1)-mimosine, 1-(2-chloroethyl)-3-(4-methylcyclohexyl)-1-nitrosourea, (8 S-ci s)-10-((3-amino-2,3,6-trideoxy-alpha-L-lyxo-hexopyranosyl)oxy)-7,8,9,10-tetrahydro-6,8, 11-trihydroxy-8-(hydroxyacetyl)-1-methoxy-5,12-naphthacenedione, 131-meta-iodobenzyl guanidine (I-131 MIBG), 5-(3,3-dimethyl-1-triazenyl)-1H-imidazole-4-carboxamide, 5-(bis(2-chloroethyl)amino)-2,4(1H,3H)-pyrimidinedione, 2,4,6-tris(1-aziridinyl)-s-thiazine, 2,3,5-tris(1-aziridinyl)-2,5-cyclohexadiene-1,4-dione, 2-chloro-N-(2-chloroethyl)-N-methyl ethanamine, N,N-bis(2-chloroethyl)tetrahydro-2H-1,3,2-oxazaphosphorin-2-amine-2-oxide, 3-deazauridine, 3-iodobenzylguanidine, 5,12-naphthacenedione, 5-azacytidine, 5-fluorouracil, (1 aS, 8 S, 8aR, 8b S)-6-amino-8-(((aminocarbonyl)oxy)methyl)-1,1a,2,8,8a,8b-hexahydro-8a-methoxy-5-methylazirino(2′,3′:3,4)pyrrolo(1,2-a)indole-4,7-dione, 6-azauridine, 6-mercaptopurine, 8-azaguanine, and combinations thereof.

Preferred chemotherapeutic agents include e.g. doxorubicin, pemetrexed, a platinum-based drug such as oxaliplatin, cisplatin or carboplatin, paclitaxel, tamoxifen, vincristine, and vinblastine.

Cytokine therapy can also be used, e.g. anti-VEGF mAbs such as bevacizumab, anti-TNFα, anti-IL-6 or anti-TGF-β. Antibodies directed to other isoforms of IL-17, e.g. IL-17A as taught in WO2014/001368, or to EGFR/HER as taught in WO2017/194554 are also of interest.

Infectious diseases are usually treated with antiviral or antimicrobial (e.g. antibiotics) agents. As an example and merely to illustrate the spirit of the invention, the treatment of fungal sepsis, e.g. mucormycosis, can be handled by further administering an antifungal agent such as posaconazole and/or amphotericin, and possibly an immunoadjuvant such as interferon-γ.

As further examples, drugs which can be used together with the combination of the invention, especially for treating tuberculosis, are: isoniazid (INH), rifampin/Rifampicin (RIF), ethambutol (EMB) and pyrazinamide (PZA), alone or in association.

As further examples, drugs which can be used together with the combination of the invention, especially for treating HIV infections, are antiretroviral therapies including:
Nucleoside Reverse Transcriptase Inhibitors (NRTIs) such as abacavir (abacavir sulfate), emtricitabine (FTC), lamivudine (3TC), tenofovir disoproxil fumarate (TDF), zidovudine (azidothymidine, AZT or ZDV);
Non-Nucleoside Reverse Transcriptase Inhibitors (NNRTIs) such as doravirine (DOR), efavirenz (EFV), etravirine (ETR), nevirapine (NVP), rilpivirine (rilpivirine hydrochloride or RPV);
Protease Inhibitors (PIs) such as atazanavir (atazanavir sulfate or ATV), darunavir (darunavir ethanolate or DRV), fosamprenavir (fosamprenavircalcoium or DRV), ritonavir (RTV), saquinavir (saquinavir mesylate or SQV), tipranavir (TPV);
Fusion Inhibitors such as enfuvirtide (T-20);
CCR5 Antagonists such as maraviroc (MVC);
Integrase Inhibitors such as dolutegravir (dolutegravirsodium or DTG), raltegravir (raltegravir potassium or RAL);
Post-Attachment Inhibitors such as ibalizumab;
Pharmacokinetic enhancers such as cobicistat (COBI).

Such medicine can be used in combination, e.g. as follows:
abacavir and lamivudine;
abacavir, dolutegravir and lamivudine;
abacavir, lamivudine and zidovudine;
atazanavir and cobicistat;
bictegravir, emtricitabine and tenofovir alafenamide fumarate;
darunavir and cobicistat;
darunavir, cobicistat, emtricitabine and tenofovir alafenamide fumarate;
dolutegravir and rilpivirine;
doravirine, lamivudine and tenofovir disoproxil fumarate;
efavirenz, emtricitabine and tenofovir disoproxil fumarate;
efavirenz, lamivudine and tenofovir disoproxil fumarate;
elvitegravir, cobicistat, emtricitabine and tenofovir alafenamide;
elvitegravir, cobicistat, emtricitabine and tenofovir disoproxil fumarate;
emtricitabine, rilpivirine and tenofovir alafenamide;
emtricitabine, rilpivirine and tenofovir disoproxil fumarate;
emtricitabine and tenofovir alafenamide;
emtricitabine and tenofovir disoproxil fumarate;
lamivudine and tenofovir disoproxil fumarate;
lamivudine and zidovudine;
lopinavir and ritonavir.

As further examples, drugs which can be used together with the combination of the invention, especially for treating HBV/HCV infections, are:
for HBV: entecavir, lamivudine (3TC), adefovir dipivoxil, interferon alpha-2b, pegylated interferon, telbivudine, tenofovir alafenamide, tenofovir;
for HCV: ribavirin, daclatasvir, sofosbuvir and velpatasvir, ledipasvir and velpatasvir, telaprevir, interferon alphacon-1, interferon alpha-2b, glecaprevir and pibrentasvir, simeprevir, pegylated interferon, pegylated interferon alpha-2b, interferon alpha-2a, sofosbuvir, ombitasvir and paritaprevir and ritonavir, boceprevir, ombitasvir and paritaprevir and ritonavir and dasabuvir, elbasvir and grazoprevir.

### REFERENCES

Throughout this application, various references describe the state of the art to which this invention pertains. The disclosures of these references are hereby incorporated by reference into the present disclosure.

The invention will be further illustrated by the following figures and examples. However, these examples and figures should not be interpreted in any way as limiting the scope of the present invention.

### FIGURES

FIG. 1. In vivo study of an anti-CTLA4 therapy in IL-17B WT vs KO mice in the B16F10 melanoma model

Tumor growth (A) and percent survival (Kaplan Meier survival curve; B) in WT and IL-17B KO mice treated with anti-CTLA4 antibody (α-CTLA4)

*** p<0.001

FIG. 2. In vivo study of anti-CTLA4 and anti-IL-17B therapies in the B16F10 melanoma murine model

Tumor growth (A and B) and percent survival (Kaplan Meier survival curve; C) in mice treated with anti-IL-17B antibody (α-IL-17B) or corresponding control antibody (IC), possibly in combination with anti-CTLA4 antibody (α-CTLA4) or corresponding control antibody (IC2)
*, p<0.05
**, p<0.01
****, p<0.0001

FIG. 3. In vivo study of an anti-CTLA4 therapy in IL-17B WT vs KO mice in the MC38 colon cancer model

Tumor growth (A and B) and percent survival (Kaplan Meier survival curve; C) in WT and IL-17B KO mice treated with anti-CTLA4 antibody (α-CTLA4) or corresponding control antibody (IC)
** p<0.01
*** p<0.001

### EXAMPLES

### Example 1/Efficacy of an Anti-CTLA4 Immunotherapy in the B16F10 Melanoma Model in the Presence (WT Mice) or Absence (IL-17B KO Mice) of IL-17B

### 1-1 Materials and Methods

7-8-week-old C57BL6 WT mice and 7-8-week-old C57BL6 IL-17B KO mice were subcutaneously grafted with 5.10⁴ B16F10 cells, 10 WT and 9 KO animals per group. Tumor growth was monitored using a caliper. At day 6, mice were treated with anti-CTLA4 antibody (i.p. injections, 200 μg/mouse, BioXCell, 9H10) and then twice a week at day 9, 13, 17, 20 and 24. Animals were sacrificed when tumors reached 1500 mm³ or in case of ulceration.

### 1-2 Results and Conclusion

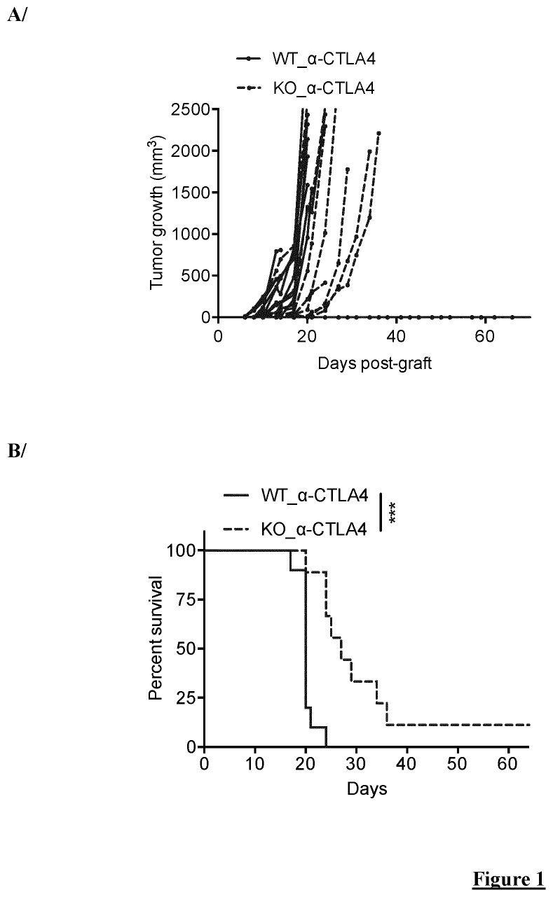

FIG. 1 shows the results obtained in terms of tumor growth (A) and percent survival (B).
The B16F10 melanoma model is markedly resistant to anti-CTLA4 therapy, which had no effect in WT animals. In sharp contrast, the response to anti-CTLA4 mAb in the IL-17B KO background was significantly improved with significantly improved mouse survival (p<0.001), including 1 complete response (11%).
In this difficult-to-treat and markedly resistant B16F10 melanoma model, the treatment with anti-CTLA4 antibody was improved in the IL-17B KO background, whereas anti-CTLA4 mAb alone had no effect.

### Example 2/Efficacy of Anti-CTLA4 and Anti-IL-17B Immunotherapies in the B16F10 Melanoma Model

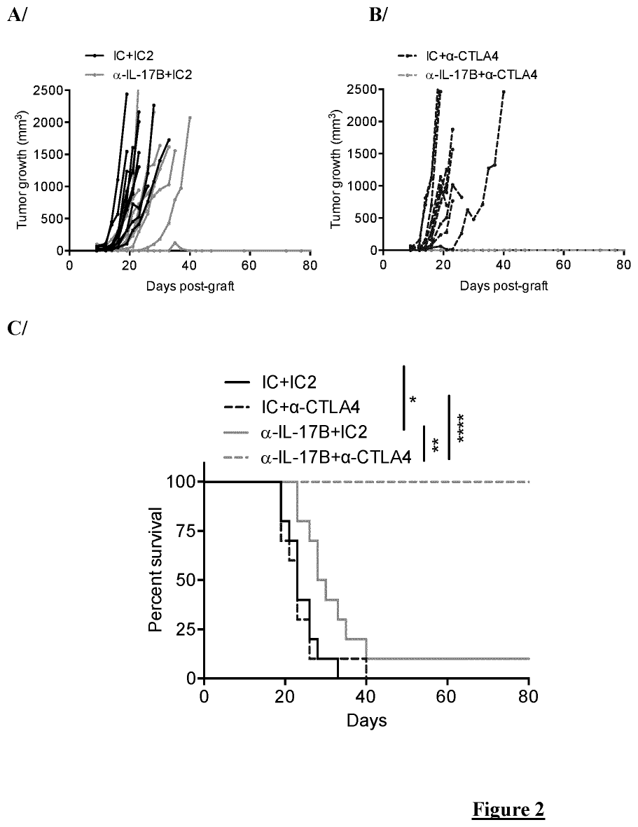

In order to confirm the results obtained in example 1 wherein the absence of IL-17B was obtained by the use of an IL-17B KO mouse, the experiment was repeated in WT mice treated with an anti-IL-17B antibody (FIG. 2).

### 2-1 Materials and Methods

8-week-old C57BL6 mice were subcutaneously grafted with 5.10⁴ B16F10 cells, 10 animals per group. Tumor growth was monitored using a caliper. At day 5, mice were treated with anti-IL-17B antibody (i.t. injections, 200 μg/mouse) or control monoclonal mouse IgG1 antibody (i.t. injections, 200 μg/mouse, RD-Biotech clone B-D38), then four times during the first week and three times a week thereafter. At day 6, mice were treated with anti-CTLA4 antibody (i.p. injections, 200 μg/mouse, BioXCell, 9H10) or control polyclonal syrian hamster antibody (i.p. injections, 200 μg/mouse, BioXCell, BP0087) and then twice a week at day 9, 12, 16, 19 and 23. Animals were sacrificed when tumors reached 1500 mm³ or in case of ulceration.

### 2-2. Results and Conclusion

As shown previously, the B16F10 melanoma model is markedly resistant to anti-CTLA4 therapy, which had no effect. Treatment with an anti-IL-17B antibody alone did induce some responses, including 1 complete responder and long-term survivor. Strikingly and in line with the data obtained using the IL-17B KO mice, the combination of anti-IL-17B antibody and anti-CTLA4 antibody induced complete tumor eradication in all animals (10/10) that remained cleared from their tumor and long-term survivors.
In this difficult-to-treat and markedly resistant B16F10 melanoma model, the treatment with anti-IL-17B antibody combined with anti-CTLA4 antibody was able to induce complete tumor regressions in all animals, whereas anti-CTLA4 mAb was unable to induce responses in this model.

### Example 3/Efficacy of an Anti-CTLA4 Immunotherapy in the MC38 Colon Cancer Model in the Presence (WT Mice) or Absence (IL-17B KO Mice) of IL-17B

In order to confirm the results obtained in example 1 in a melanoma model, the experiment was repeated in a colon cancer model.

### 3-1. Materials and Methods

10-week-old C57BL6 WT mice and 9-11-week-old C57BL6 IL-17B KO were subcutaneously grafted with 5.10⁵ MC38 cells, 10 WT and 8 or 9 KO animals per group. Tumor growth was monitored using a caliper. At day 6, mice were treated with anti-CTLA4 antibody (i.p. injections, 200 μg/mouse, BioXCell, 9H10) or control polyclonal syrian hamster antibody (i.p. injections, 200 μg/mouse, BioXCell, BP0087), and then twice a week at day 9, 12, 15, 19, 23, 27, 30. Animals were sacrificed when tumors reached 1500 mm³ or in case of ulceration.

### 3-2. Results and Conclusion

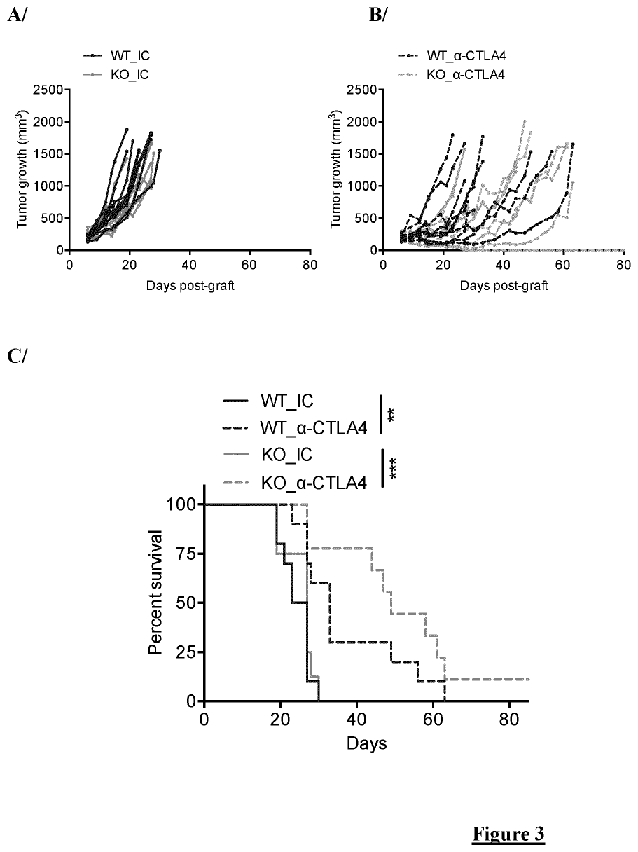

As previously observed, the data shown on FIG. 3 reveal that the effect of the anti-CTLA4 antibody is improved in the KO background.
Anti-CTLA4 therapy was able to induce some transient responses in the WT mice, yet no complete responses were obtained and none of the animals survived. In the IL-17B KO background, the treatment with the anti-CTLA4 mAb was improved with 1 mouse out of 9 (11%) that achieved a complete response, remained tumor cleared and survived on the long term.
This example validates that the CTLA4 and IL-17B inhibitory approach according to the invention can be applied to other types of cancer or even to other diseases.

1. A composition comprising a CTLA4 inhibitor and an IL-17B inhibitor.

2. (canceled)

3. A method for treating a cancer or an infectious disease in a subject, comprising administering to the subject the composition according to claim 1.

4. A method of treating a cancer or an infectious disease in a subject, comprising administering to the subject a composition comprising an IL-17B inhibitor, wherein the subject has been treated with a CTLA4 inhibitor.

5. The method according to claim 4 wherein administering the composition comprising the IL-17B inhibitor increases the sensitivity of the subject to the CTLA4 inhibitor.

6. The method according to claim 4 wherein the subject is resistant to the treatment with the CTLA4 inhibitor.

7. The method according to claim 3, wherein the infectious disease is selected in the group consisting of severe sepsis, septic shock, viral infections, fungal infections, mosquito-borne infectious diseases, and bacterial infections.

8. The method according to claim 7 wherein the infectious disease is selected in the group consisting of human immunodeficiency virus, simian immunodeficiency virus, hepatitis, HBV, and malaria.

9. The method according to claim 3 wherein the cancer is resistant to CTLA4 inhibitors.

10. The method according to claim 3 wherein the cancer is selected from the group consisting of melanoma; Renal Cell Carcinoma; ColoRectal Cancer; Small Cell Lung Cancer; Non-Small Cell Lung Cancer; esophageal cancer; breast cancer; Hepatocellular Carcinoma; thyroid cancer; pancreatic cancer; ovarian cancer; Myelodysplastic Syndrome; Acute Myeloid Leukemia; Diffuse Large B-Cell Lymphoma; glioblastoma; sarcoma; Soft tissue Sarcoma; nasopharyngeal carcinoma; mesothelioma; head and neck cancer; prostate cancer and gastrointestinal cancer.

11. The composition according to claim 1 wherein the CTLA4 inhibitor is an inhibitor of CTLA4 or of B7-1 or of B7-2.

12. The composition according to claim 11, wherein the CTLA4 inhibitor is ipilimumab or tremelimumab.

13. The composition according to claim 1, wherein the IL-17B inhibitor is an antibody directed against IL-17B or an antibody directed against a receptor of IL-17B (IL-17RB).

14. The composition according to claim 1 wherein the CTLA4 inhibitor or IL-17B inhibitor is an inhibitor of CTLA4 or IL-17B expression.

15. The composition according to claim 11, wherein the CTLA4 inhibitor is an anti-CTLA4 antibody.

16. The composition according to claim 14, wherein the CTLA4 inhibitor or IL-17B inhibitor is a siRNA or an antisense oligonucleotide.

17. The method according to claim 7, wherein the viral infection is due to human immunodeficiency virus, simian immunodeficiency virus, hepatitis virus, cytomegalovirus or Epstein-Barr virus.

18. The method according to claim 8, wherein the infectious disease is HBV.

19. The method according to claim 7, wherein the fungal infection is mucormycosis.

20. The method according to claim 7, wherein the mosquito-borne infectious disease is malaria.

21. The method according to claim 7, wherein the bacterial infection is tuberculosis.
